(19) United States
(12) Patent Application Publication
(10) Pub. No.: US 2002/0052491 A1
(43) Pub. Date: May 2, 2002

(54) Method for the production of 2-chloro-2' -deoxyadenosine (cladribine) and its 3,5-di-O-p-toluoyl derivative

(76) Inventors: Szepsel Gerszberg, Buenos Aires (AR); Daniel Alonso, Buenos Aires (AR)

Correspondence Address: Miguel Ortiz, Sterrenbeld Biotechnologie, North America, Inc., Constitucion 4234, Buenos Aires, 1254, AR

(21) Appl. No.: 09/982,805

(22) Filed: Oct. 22, 2001

(30) Foreign Application Priority Data
Apr. 28, 1999 (US) PCT/US99/09158

(51) Int. Cl. C07H 19/16

(52) U.S. Cl. 536/27.21

(57) ABSTRACT

A process for the production of cladribine, 2-chloro-2′-deoxyadenosine, is provided which involves the direct glycosylation of 2-chloro-6-aminopurine with 1-chloro-2-deoxy-3,5-di-O-p-toluoyl-α-D-erythropentofuranose. The process is carried out by first forming the sodium salt of 2-chloro-6-aminopurine and allowing the sodium salt to react with 1-chloro-2-deoxy-3,5-di-O-p-toluoyl-α-D-erythropentofuranose in the presence of a moderately polar solvent such as acetone. The final product, cladribine, is produced by removal of the p-toluoyl groups by the action of methanolic ammonia or methanolic sodium methoxide.

### BACKGROUND OF THE INVENTION

1. Technical Field

The present invention relates generally to a method of producing 2-chloro-2′-deoxyadenosine (2-CdA; cladribine, I).

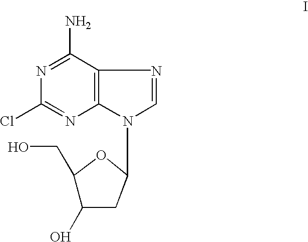

More particularly, the invention relates to a method of producing 2-CdA by the glycosylation of the sodium salt of 2-chloro-6-aminopurine with 1-chloro-2-deoxy-3,5-di-O-p-toluoyl-α-D-erythropentofuranose (II), followed by isolation of the 3,5-di-O-p-toluoyl derivative and removal of the p-toluoyl protecting groups.

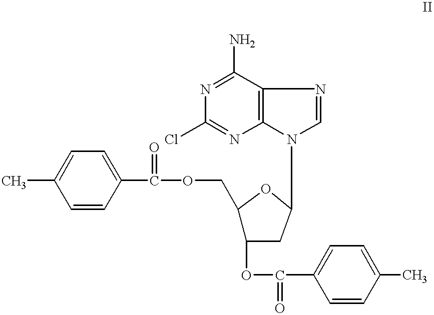

2. Related Art

Cladribine is useful as an antileukemic agent, i.e., in the treatment of leukemia, such as hairy cell leukemia. 2-CdA is also known to have immunosuppressive activity. Many processes for preparing 2-CdA and similar compounds are known which involve the direct condensation or coupling of a heterocycle (e.g., purine, pyrimidine, imidazole) with an activated, conveniently protected sugar at C-1.

The first synthesis of 2-CdA was described by Ikehara and Tada as an intermediate in the preparation of 2′-deoxydenosine. (Ikehara. M. et al, J. Am. Chem. Soc. 12(17):2344-2345 (1963); Id. 87(3):606-610 (1965)). The starting material (2,8-dichloroadenine), the use of the chloromercury salt of the purine base in the condensation step, the sugar employed (2-O-acetyl-3-O-tosyl-5-O-methoxycarbonyl-D-xylofuranosyl chloride), as well as the deoxygenation process at C-2′ of this method, are different from all of the methods hereinafter described.

A second synthesis was published by Christensen et al. based on the fusion coupling of 2,6-dichloropurine with 1,3,5-tri-O-acetyl-2-deoxy-D-erythropentofuranose resulting in an anomeric mixture of the acetyl-protected deoxynucleosides. (Christensen, L. F. et al., J. Med. Chem. 15(7):735-739 (1972). When the resulting mixture of this coupling was treated with liquid ammonia, the clean ammonolysis of the chlorine atom at C-6 and deacetylation took place and 2-chloro-2′-deoxy-9-α-adenosine and 2-chloro-9-β-adenosine were obtained. This mixture was then reacylated with p-toluoyl chloride and the new blocked mixture was finally resolved by chromatography. Removal of the p-toluoyl groups with methanolic sodium methoxide at room temperature resulted in a mixture of 2-chloro-2′-deoxyadenosine and its α-anomer with a total yield of 16% and 9%, respectively. Fusion of 2,6-dichloropurine with methyl 3,5-di-O-p-toluoyl-2-deoxy-D-erythropentofuranoside gave a mixture of blocked deoxyribonucleosides which was separated by silica gel chromatography. Treatment of each anomer with liquid ammonia was used as an alternative route and deprotection of the toluoyl groups and simultaneous substitution at C-6 took place resulting in 2-chloro-2′-deoxyadenosine (I) with a total yield of 8%, while the yield for the α-anomer was 13%.

The synthesis of biologically active 9-β-purine-2-deoxyribonucleosides commonly involves direct glycosylation of the heterocycle with an activated 2-deoxyribose derivative. Glycosylation procedures introducing the 2-deoxy-β-D-ribofuranosyl moiety into an aglycon invariably provide anomeric mixtures as well as positional isomers. Not only is the yield of the desired product low in such reactions, but purification is often difficult due to the similar mobility of isomeric products during chromatographic separation. Such a process is both time consuming and costly.

The third synthesis of 2-CdA was postulated by Kazimierczuk et al and Robbins et al. and minimizes the intrinsic difficulties caused by methods involving direct glycosylation. (Kazimierczuk, Z. et al., J Am. Chem. Soc. 106(21):6379-6382 (1984); Robbins, R. K. et al., Eur. Patent No. 173059 (1985)). This coupling method comprises the use of chloroheterocyclic derivatives and a protected 1-α-chloro-2-deoxyribose. This method provides a clear improvement in reaction selectivity.

The respective sodium salts of the bases to be glycosylated were prepared in situ using sodium hydride in acetonitrile. This solution was made to react in the same medium and at room temperature with 1-chloro-2-deoxy-3,5-di-p-O-toluoyl-α-D-erythropentofuranose (III), having the formula

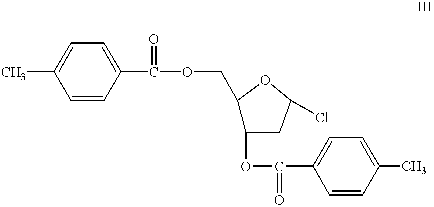

The α-configuration of the starting sugar in the solid state facilitates the formation of the blocked 2-deoxy-β-nucleosides during the anionic attack of the heterocyclic nitrogen at the C-1 site. The formation of the β-anomer is attributed to a direct Walden inversion (S_{N}2). This sodium salt glycosylation procedure is considerably superior to those reported, including silyl derivatives, molecular sieves and phase transfer catalysis.

In contrast to ribofuranose synthons which possess an acyloxy substituent at C-2 that directs base attack on the β face of the sugar, glycosylation with 2-deoxyribofuranoses may result in the formation of α and β anomeric products, due to the diminished steric control exhibited by deoxyribofuranoses. In fact, the non-desired N-7 coupling product evidentiates the loss of regioselectivity resulting from the ambidentate character of the anion. Hildebrand et al. described for this particular case the formation of 9-β-(50%), 7-β-(15%) and 9-α-(1.5%) nucleosides, while Kazimierczuk et al. reported a 59% and 13% yield for the first two nucleosides (9-β- and 7-β-), respectively.

Purification by column chromatography is often difficult due to the quite similar mobility of these products which lowers the yield and makes the isolation of pure fractions dificult. Pure 2,6-dichloro-9-(2′-deoxy-3,5-di-O-p-toluoyl-β-D-erythropentafuranosyl) purine was ammonolysed with methanolic ammonia at 100° C. giving 2-chloro-2′-deoxyadenosine (I) which must be purified by a silica gel column. The total yield of isomer 9-β was 42%.

A fourth process for preparing 2-CdA was described by Chen. (Chen, R. H. K., U.S. Pat. No. 5,208,327). This process differs from the previous ones by employing guanosine, whose β-glycoside bond is between C-1 of the ribose and N-9 of the guanine moiety, as a starting material. The main disadvantages of this method are the low total yield (2.75%) and the complexity of the method which involves eight steps with four chromatographic purification steps of certain intermediates and an additional chromatographic step to obtain the final product, cladribine.

A fifth preparation method is an enzymatic synthesis achieved by direct transfer of the 2-deoxyribofuranose moiety from 2′-deoxythymidine to 2-chloroadenine catalyzed by purified trans-N-deoxyribosylase obtained from α E. Coli BMT-1D/1A strain. (Mikhailopulo, I. A. et al., Nucleosides & Nucleotides 12(3-4):417-422 (1993)). This method is highly stereospecific, but the reaction scale is limited to the production of less than 1 millimole of 2-CdA.

Accordingly, the above-discussed prior art describes the preparation of 2-chloro-2′-deoxyadenosine based on the direct glycosylation of 2,6 dichloropurines and further dehalogenation with liquid ammonia or methanolic ammonia. Because of the above-described disadvantages of this method there is a need for the synthesis of 2-chloro-2′-deoxyadenosine by means of direct glycosylation of 2-substituted adenines.

### SUMMARY OF THE INVENTION

The present invention relates to the synthesis of 2-chloro-2′-deoxyadenosine by direct glycosylation of 2-chloro-6-aminopurine. The principal advantage of this method is that the intermediate and final products obtained do not require column chromatography purification, facilitating the synthesis of pure cladribine. Another of the advantages of this method is the suprisingly high yield of pure cladribine obtained.

Accordingly, it is an aspect of the present invention to provide a novel procedure for the preparation 2-chloro-2′-deoxyadenosine having the formula

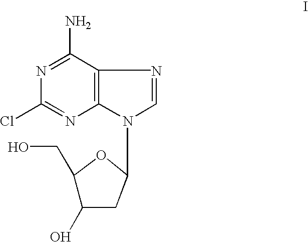

comprising:

(a) glycosylating 2-chloro-6-aminopurine having the formula

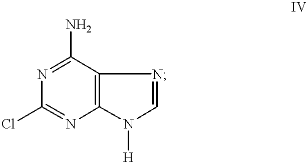

as its sodium salt, having the formula

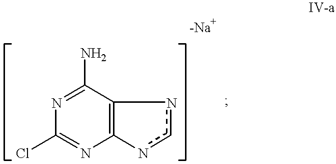

with 1-chloro-2-deoxy-3,5-di-O-p-toluoyl-α-D-erythropentofuranose having the formula

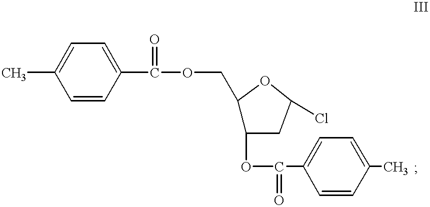

(b) isolating the resulting glycoside having the formula

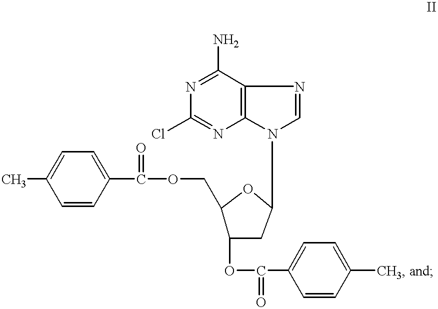

(c) removing the p-toluoyl groups to obtain 2-chloro-2′-deoxyadenosine (I).

### DETAILED DESCRIPTION OF THE PREFERRED EMBODIMENTS

The starting material, 2-chloro-6-aminopurine (IV), is prepared from 2,6-dichloropurine according to Brown et al. (Brown, G. B. et al., J. Org. Chem. 23:125-126 (1958)).

Since the H-9 proton of adenine is known to be sufficiently acidic (pKa=9.80) the sodium salt (IVa) is prepared by reacting (IV) with one equivalent of methanolic sodium methoxide, instead of sodium hydride in acetonitrile. Excess sodium methoxide must be avoided because it is harmful to the coupling reaction with the protected sugar III.

It is known to those of skill in the art that solvent effects can provide good anomeric specificities in moderately polar solvents. As used herein, the term “moderately polar solvent” refers to a solvent with a dielectric constant at 20-25° C. between about 6 and about 25. A preferred solvent is anhydrous acetone. Accordingly, after removal of the solvent used to produce the sodium salt (IVa), methanol, the sodium salt of 2-chloro-6-aminopurine (IVa) was obtained and used directly for the coupling reaction with the 1-chlorosugar (III) by suspending it in anhydrous acetone.

A prerequisite for the successful formation of high yields of β-2-deoxynucleosides, assuming that a bimolecular nucleophilic substitution (S_{N}2) reaction mechanism can be invoked, is the prevention of anomerization of the α-chlorosugar III. Hubbard et al. studied the stability of this α-chlorosugar in solvents having a range of dielectric constants. (Hubbard, A. J. et al., Nucl. Acids Res. 12(7):6827-6837 (1984)). As expected, much more β-chlorosugar was formed in polar solvents like acetonitrile (70% in 2 hours) than in chloroform (20% in 2 hours). In benzene, no anomerization was detectable. Chloroform and benzene are inadequate solvents for nucleoside synthesis by this route, due to the insolubility of sodium salts in non-polar solvents. In addition, it is known that these solvents do not promote nucleophilic substitution.

The protected 1-chloro-2-deoxyribose (III) has the α-configuration in the solid state, but in solution undergoes an equilibrium process which results in a mixture composed mostly of the α-anomer with small quantities of the β-anomer. Accordingly, reaction conditions which prevent the α-anomer from having enough time to undergo such anomerization should be chosen.

Preferential formation of β-anomers in reactions with 1-α-chlorosugars may result if the rate of glycosylation is much faster than the sugar anomerization or if the 1-β-chloro sugar is unreactive. The use of acetone as solvent favors the preferential formation of the 9-β-nucleoside.

In these sodium salt glycosylation procedures, the attack of the ambidentate purine anion on the 1-α-chlorosugar induces 7-α and 7-β nucleoside formation. A systematic study of 2-deoxyribonucleoside isomer distribution from this procedure of substituted purines at C-6 (H, Cl, Br, SCH₃) was studied by Hildebrand et al; in each case, 9-β-nucleoside always prevails, but 9-α, 7-α and 7-β isomer ratio vary with the C-6 substituent (Hildebrand, C. et al., J. Org. Chem. 57(6):1808-1813 (1992)).

In this invention the use of 2-chloro-6-amino-purine in a sodium salt glycosylation reaction is described for the first time.

A similar glycosylation of the sodium salt of 2,6-dichloropurine results in a mixture of 9-β and the corresponding 7-β and 9-α-isomers, which are dificult to separate on a silica gel column. In our case, the main product of the reaction is accompanied only by traces of other compounds of similar mobility and by unreacted chlorosugar starting material and degradation products of the chlorosugar.

Therefore, the presence of an amino group at C-6 directs a more selective attack by the N-9 anion on the β-face of the α-chlorosugar, forming mainly the 9-β-nucleoside. This high regioselectivity and anomeric specificity is achieved in a moderately polar solvent such as acetone.

It should be recalled that the 1-chlorosugar is easily anomerized in polar solvents and on the other hand, the purine sodium salt is insoluble in non-polar organic solvents. Therefore, in a moderately polar solvent such as acetone a good anomeric specificity is achieved though its yield is not very high, probably due to the low solubility of the sodium salt and/or sugar degradation.

The coupling reaction preferably takes place in anhydrous acetone at room temperature under a nitrogen atmosphere.

The best results (yield 60%) were obtained when the reaction was carried out at approximately a 2:1 molar ratio of sodium salt of 2-chloroadenine (IVa) to 1α-chlorosugar (III). Under these conditions, the reaction proceeds to completion in about 2 hours.

The solution is evaporated to dryness and the residue obtained is substantially pure as demonstrated by thin layer chromatography. The product, 2-chloro-6-amino-9-(3,5-di-O-p-toluoyl-2-deoxy-β-D-erythropentofuranosyl) purine (II), is crystallized from ethanol. The melting point of the intermediate product (II) agrees with that described for the intermediate product synthesized by another pathway.

Ammonolysis of both p-toluoyl groups may be conducted at room temperature, either by the action of methanolic ammonia or methanolic sodium methoxide. In the event methanolic ammonia is used, the work up consists of solvent removal by evaporation and then treatment of the resulting residue with n-hexane to remove methyl p-toluate and p-toluamide. When methanolic sodium methoxide is employed, the solution must be neutralized, preferably with an ion exchange resin (cationic form), and then treated with n-hexane. The yield of this step including ammonolysis of the protecting groups and purification, is 70% and the total yield starting from 2-chloro-6-aminopurine is 42%. This method is advantageous in that it does not need additional purification of the intermediate product II or the final product I through chromatographic columns.

The residue obtained is 2-chloro-2′-deoxyadenosine, impurified with traces of other products. By recrystallization from an ethanol-water mixture, a chromatographically pure product is obtained having a melting point, specific optical rotation, ¹H NMR, ¹³C NMR, mass and UV spectra identical to those reported in the literature for cladribine.

### In Vitro Activity of Cladribine

The in vitro effect of cladribine (2-CdA) has been studied on a number of cell lines, showing different degrees of sensitivity, represented by the IC₅₀ value (concentration required to inhibit 50% of growth) on a case-by-case basis.

Cladribine shows in vitro cytotoxicity against cell lines derived from human cells like T lymphocytes, B lymphocytes, non-T/non-B lymphocytes and myeloblastoid lines (Avery, T. L. et al., Cancer Res. 49:4972-4978 (1989); Carson, D. A. et al., Proc. Nat'l Acad. Sci. (USA) 77:6865-6869 (1980); Wataya, Y. et al., Adv. Exp. Med. Biol. 253(B):227-234 (1989)). IC₅₀ values for cladribine range between 3-338 nmol/L for T cells, between 6-67 nmol/L for B cells, and between 5-70 nmol/L for myeloid cells (Avery, T. L. et al., Cancer Res. 49:4972-4978 (1989)). Concentration of cladribine necessary to obtain an inhibitory effect depends on the cell type and cell pathogenic condition. The 5 nmol/L concentration inhibits the thymidine uptake in lymphoblastoid leukemia cells and has no effect on the granulocyte and macrophage colony formation in normal bone marrow. (Carson, D. A. et al., Blood 62(4):737-743 (1983).

Cladribine has low activity on multiple myeloma cells (Nagourney, R. A. et al., Br. J. Cancer 67:10-14 (1993)) and cell lines of bone tumors (Carson, D. A. et al., Blood 62(4):737-743 (1983)). Activity reported for some human melanoma cell lines was IC₃₇ at 12-22 nmol/L (Parson, D. G. et al., Biochem. Pharm. 35:4025-4029 (1986)) and for a neuroblastoma cell line, IC₅₀ at 60 nmol/L (Avery, T. L. et al., Cancer Res. 49:4972-4978 (1989)).

Petzer et al. investigated the effects of cladribine on clonal growth of hematopoietic progenitor cells. (Petzer, A. L. et al., Blood 78:2583-2587 (1991)). Cladribine inhibits both myeloid progenitor cell growth and T lymphocyte colony forming cells on a dose-dependent basis. Erythroid progenitor cells show a dose-dependent sensitivity to cladribine, and this sensitivity decreases as the maturation state of said progenitors rises. Cladribine has higher effect on progenitor cells in the early maturation state; IC₅₀ values for more immature progenitors (granulocyte-macrophage colony forming units and erythrocyte burst forming units) are of approximately 16 to 20 nmoles/L. Mature erythroid colony forming units (mBFU-E) show an IC₅₀ value of 38 nM. More differentiated erythroid colony forming cells (CFU-E), show an IC₅₀ value of 56 nM. A higher concentration is needed for a complete inhibition of lymphoid colony formation (1280 nmol/L). Cladribine is active both in dividing and non-dividing cells (Carson, D. A. et al., Proc. Nat'l Acad. Sci. (USA) 79:3848 (1982); Carson D. A. et al., Blood 62(4):737-743 (1983)). This activity distinguishes this drug from other agents that affect purine and pyrimidine metabolism (Carson D. A. et al., Blood 62(4):737-743 (1983); Carson, D. A. et al., Adv. Exp. Med. And Biol. 165(B):351-356 (1984).

Cladribine activity against T lymphocytes that are not in cell division process depends on the duration of exposure to drug. Peripheral lymphocytes incubated with cladribine 100 nmol/l during 24 hours, followed by washing, retain viability after washing, while 60% of cells die after seven-day incubation with cladribine 8 nmol/L. (Carson, D. A. et al., Proc. Nat'l Acad. Sci. (USA) 79:3848 (1982); Carson D. A. et al., Blood 62(4):737-743 (1983)).

The following synthetic examples describe the invention in greater extent particularly and are intended to be a way of illustrating but not limiting the invention.

TABLE Number 1
Summary of in vitro Sensitivity to Cladribine of
Different Cell Lines and Types.

| CELL LINE | IC₅₀ (nmol/L)** |
|---|---|
| Acute lymphoblastic leukemia* | 34 |
| Acute myelogenous leukemia* | 20 |
| Chronic lymphocytic leukemia* | 39 |
| Hairy cell leukemia* | 31 |
| Multiple myeloma* | 1750 |
| Non-Hodgkin's lymphoma* | 87 |
| T cells # | 3-338 |
| B cells # | 6-67 |
| Normal bone marrow ° | 15 |
| Epstein Barr B lymphoblastoid cell lines | 200 |
| DAUDI cell line & | 210 |

**IC₅₀ Drug concentration required to inhibit 50% of growth.
*Cladribine (Bryson, H. M. et al., Drugs 46(5):872-894 (1993))
# Avery, T. L. et al., Cancer Res. 49:4972-4978 (1989)
° Carson D. A. et al., Blood 62(4):737-743 (1983) & Data Bio Sidus

### EXAMPLES

### General Methods

Melting points were taken on a Mel-Temp II apparatus and are uncorrected.

NMR spectra were recorded on a Brucker MSL 300 spectrometer at ¹H (300.1 MHz) and ¹³C (75.47 MHz). Chemical shifts are reported in parts per million (δ) relative to internal tetramethylsilane and deuterated dimethylsulfoxide as the solvent.

Mass spectra were recorded on a Quatro II-Micromass instrument using positive ion electrospray.

Infrared spectra were recorded as KBr pellets on a Nicolet-Magna 560 FT-IR spectrophotometer.

Ultraviolet spectra were recorded using a Shimadzu UV 1603 spectrophotometer.

Optical rotations were determined using a Perkin-Elmer Model 343 polarimeter with a 1 cm³, 1 dm cell.

Evaporations were carried out with a Büchi Rotavapor R-114.

TLC was performed using aluminium-backed sheets of silica gel 60 F₂₅₄. Spots were visualized under 254 nm UV light.

HPLC assays were carried out with a Waters 600 E high-pressure liquid chromatographer with 214 nm detection (Waters 991); a Merck-Lichrocart column in the reverse phase mode (Cartridge licrhospher 100 RP 18 (4×25 mm) and a linear gradient mobile phase of 0.1 M NH₄CH₃CO₂ (pH 6.55) and CH₃CN, and a flow rate of 1.2 min.

### Example 1

### Preparation of 2-Chloro-6-amino-9-(3,5-di-O-p-toluoyl-2-β-D-erythropentofuranosyl)-purine (II)

A solution containing 7.2 g (58.9 mmole) of sodium methoxide in 300 ml of methanol was added to a suspension of 22.5 g (133 mmole) of 2-chloro-6-aminopurine in 260 ml of methanol. The mixture was magnetically stirred for 20 minutes at room temperature.

The solvent was then removed from the reaction mixture under reduced pressure in a rotavapor not exceeding the bath temperature 55 ° C.

Then 350 ml of anhydrous acetone was added and removed as indicated.

The resulting residue of 2-chloro-6-aminopurine sodium salt, was suspended in 1,300 ml of anhydrous acetone and afterwards 22.9 g (58.9 mmole) of 1-chloro-2-deoxy-3,5-di-O-p-toluoyl-α-D-erythropentofuranose was added.

The mixture was stirred at room temperature under a nitrogen atmosphere for 2 hours. The solution was then filtered and the filtrate evaporated to dryness. The residue was then dissolved in boiling ethanol (1,100 ml) and decolorized with activated carbon (2.2 g).

The clear solution was cooled overnight, filtered, washed with cold ethanol (3×20 ml) and dried in a vacuum oven at 40° C., to give the title compound (18.5 g, 60.2%), mp 190-192° C. with softening at 170° C. (Lit.: Christensen et al., 192-194° C., (s) 170-180° C.); TLC (CHCl₃-Acetone 4:3): R_{f} 0.37.

### Example 2

### Preparation of 2-chloro-2′-deoxyadenosine. (Cladribine) (I)

A suspension of 2-chloro-6-amino-9-(3,5-di-O-p-toluoyl-2-β-D-erythropentofuranosyl)-purine (II) (5.6 g, 10.7 mmole) in methanolic ammonia (saturated at 10° C., 285 ml) was stirred at room temperature for approximately 20 h, and the reaction was monitored by TLC (CHCl₃:MeOH 9:1, R_{f} 0.68) until disappearance of the starting material.

The clear yellowish solution was evaporated to dryness and the resulting pasty residue was triturated with n-hexane (3×25 ml), heated to reflux in a mixture of ethanol-water (9.5:1; 42 ml) and decolorized with activated carbon (0.28 g).

The solution was cooled overnight, filtered, washed with cold ethanol (1×2 ml) and dried in a vacuum oven at 40° C. to give analytically pure colorless cladribine (2.15 g, 70.2%); mp 210-214° C. (softens) and then solidifies and turns brown, does not melt below 300° C.; TLC (CHCl₃:MeOH 7:3) R_{f} 0.60; UV (95% EtOH): λ max. 265 nm (E 15,600); IR (FIG. 1 +L);

¹H NMR (d₆-DMSO): δ 2.27 (m, 1H, H-2 α), 2.63 (m, 1H, H-2 β), 3.84 (m, 1H, H-4), 4.37 (m, 1H, H-3), 4.97 (t, 1H, J=5.6 Hz, OH-5), 5.32 (d, 1 H, J=4.2 Hz, OH-3), 6.24 (t, 1H, J_{1-2α}=J_{1-2β}=6.8 Hz, H-1), 7.81 (s, 2H, NH₂), 8.34 (s, 1H, H-8); ¹³C NMR (d₆-DMSO): δ 39.7 (C-2), 61.9 (C-5), 71.0 (C-3), 84.0 (C-1), 88.2 (C-4), 118.4 (C-5), 140.3 (C-8), 150.3 (C-4), 153.3 (C-2), 157.0 (C-6); Mass spectrum (electrospray): m/z (Ir) 288 ([MH+2]⁺, 33%), 286 ([MH]⁺, 95%), 172 ([MH+2-sugar]⁺, 34%), 170 ([MH-sugar]⁺, 100%); HPLC: 16.32 min; [αa]_{D}²⁶ −19.2 (C1, DMF).

What is claimed is:

1. A process for forming 2-chloro-2′-deoxyadenosine having formula I 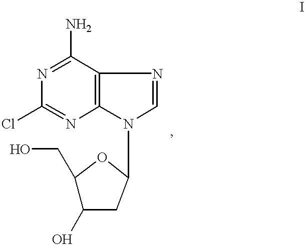
comprising:
(a) glycosylating 2-chloro-6-aminopurine having formula IV 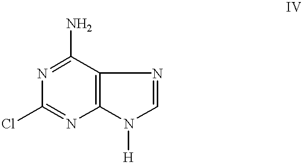
as its sodium salt, with 1-chloro-2-deoxy-3,5-di-O-p-toluoyl-α-D-erythropentofuranose having formula III 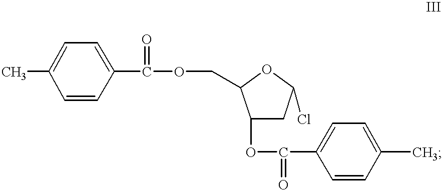
(b) isolating the resulting compound, 2-chloro-6-amino-9-(3,5-di-O-p-toluoyl-2-deoxy-β-D-erythropentofuranosyl) purine, having formula II 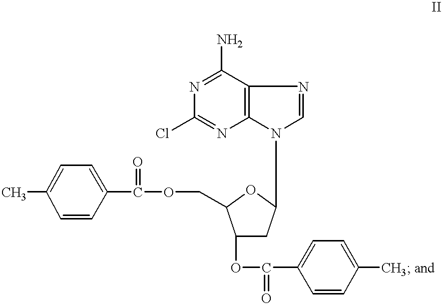
(c) removing the p-toluoyl groups from the compound of formula II, to produce 2-chloro-2′-deoxyadenosine having formula I.

2. A process according to claim 1, where a moderately polar anhydrous solvent is employed as a solvent in step (a).

3. A process according to claim 2 wherein said moderately polar solvent is acetone.

4. A process according to claim 1 where the removal of p-toluoyl groups in step (c) is carried out by the action of methanolic ammonia or methanolic sodium methoxide.

5. A process according to claim 1 where said sodium salt is formed by reaction of 2-chloro-6-aminopurine with anhydrous methanolic sodium methoxide at room temperature.

6. A process according to claim 1, wherein said sodium salt is present in approximately a 2:1 molar ratio to said 1-chloro-2-deoxy-3,5-di-O-p-toluoyl-α-D-erythropentofuranose.

7. 2-Chloro-6-amino-9-(3,5-di-O-p-toluoyl-2-deoxy-β-D-erythropentofuranosyl) purine, produced by the process of claim 1.

8. Cladribine, produced by the process of claim 1.
